(19) United States
(12) Patent Application Publication
(10) Pub. No.: US 2019/0328569 A1
(43) Pub. Date: Oct. 31, 2019

(54) Spinal Support with Adjustable Curvature

(71) Applicant: Edward Tansingco, Ontario (CA)

(72) Inventor: Edward Tansingco, Ontario (CA)

(21) Appl. No.: 15/963,646

(22) Filed: Apr. 26, 2018

(51) Int. Cl. A61F 5/02

(52) U.S. Cl. CPC A61F 5/024; A61F 2005/0197; A61F 5/028

(57) ABSTRACT

A spinal support, in particular a cervical or lumbar support, with adjustable curvature, has a hard shell with a convex curvature in a longitudinal direction generally conforming to a concavely curved region of a human spine. A stiff resilient conformal tongue on an inner surface of the hard outer shell and one end hinged to the hard shell and an opposite end free to move in a longitudinal guide permitting forcible deformation of the tongue to create an arch with varying degrees of curvature. A retaining mechanism permits locking of the opposite end so as to retain the tongue in a deformed arch configuration with a selected degree of curvature.

### FIELD OF THE INVENTION

This invention relates to the field of orthopedics, and in particular to a spinal support for promoting correct anatomical curvature in a human spine, especially the cervical and lumbar regions.

### BACKGROUND OF THE INVENTION

As illustrated in FIG. 1A, the human spine 10 in its unstressed state has a natural curvature with three curved regions, namely the concave cervical spine 12, the convex thoracic spine 14, and the lumbar spine 16. Many people suffer from a loss of normal curvature, or lordosis, in the cervical or lumbar spines from injury or from habitually prolonged poor posture. Loss of normal Lordosis in the cervical and lumbar spine creates uneven pressure causing disc and vertebral degeneration, leading to neck and back pain. Such patients may require treatment that includes reinforcement of the lordotic curve with the use of a support to restore the natural curvature. Some patients, for example, patients with a military neck or straight lumbar spine, normally cannot tolerate aggressive support immediately at the beginning of their treatment and therefore would benefit from beginning their treatment with a less severe lordotic support which can be gradually increased as they progress through their treatment.

Different patients have different sized and shaped heads, different bone structures, musculature and tissues that requires a support device to have some adjustability to accommodate their specific dimensions. The ability to adjust the severity of support is important because the required support can vary at different times throughout the patient's treatment.

For example, when a patient is being treated for a loss of lordosis in the cervical or lumbar spine, a therapist may increase lordotic support to affect a change in the curvature of the spine while under their supervision. The therapist may then recommend a decreased level of lordotic support when the patient is in recovery at home.

There are examples of devices, such as the Denneroll™, which are used in the medical field to reinforce lordotic curve to the spine. However, such devices are not easily height adjustable and require the use of additional blocks to increase support height or may be offered in specific sizes. The different sizes offered are usually small, medium or large and so the choice of support level for the patient therefore is very limited. In using such devices, the patient must make due with very abrupt transitions in level of support, which can be uncomfortable and even painful for someone who is recovering from injury.

Pneumatic inflatable supports, such as described in U.S. Pat. No. 5,382,226, or the product known as Neck-Relief™ offer very fine adjustability in the level of support they provide. However, pneumatic supports do not have accurately specific fixable gradations to determine their height, so a patient cannot accurately determine and maintain a chosen degree of support. Also, pneumatic supports can lose air so that a patient cannot rely on holding or returning to a specific position over a period of days or weeks, and in a reduced pressure environment, such as found in an airplane cabin, may tend to overinflate.

Furthermore, pneumatic supports tend to be complex in construction requiring many parts. They are therefore expensive to manufacture and service. Distributors of medical equipment complain that many products with pneumatic components fail in the field.

It has been demonstrated that a perfect arc that spans the length of the neck is not comfortable to support a patient's lordotic curve especially in the cervical spine. A wide arc such as ARC™ neck orthotic impinges on the suboccipital region of the head and also the spinous process of the T1 vertebra causing discomfort and pain.

### SUMMARY OF THE INVENTION

Embodiments of the invention provide a spinal support, in particular a cervical support or a lumbar support, with adjustable curvature.

According to the present invention there is provided a spinal support with adjustable curvature, comprising a hard shell having a convex curvature in a longitudinal direction generally conforming to a concavely curved region of a human spine; a stiff resilient conformal tongue on an inner surface of said hard shell having one end hinged to the hard shell and an opposite end free to move in a longitudinal guide permitting forcible deformation of the tongue to create an arch with varying degrees of curvature; and a retaining mechanism for locking said opposite end so as to retain said tongue in a deformed arch configuration with a selected degree of curvature.

The spinal support allows patients to benefit by beginning their treatment with a less severe lordotic support, which can then be gradually increased as they progress through their treatment by increasing the depth of the arch provided by the resilient tongue. This can be provided by a pliable, resilient material with shape memory, such as polypropylene plastic or acetal plastic.

The spinal support may be a cervical support, in which case the hard shell can be shaped into a cradle and provided with cushioning, or a lumbar support in which case the hard shell may be shaped to conform to the lower back of a human.

It has been demonstrated that a perfect arc that spans the length of the neck is not comfortable to support a patient's lordotic curve especially in the cervical spine. A wide arc impinges on the suboccipital region of the head and also the spinous process of the T1 vertebra causing discomfort and pain. A parabolic arch is preferred in the sagittal section with the peak of the arch in the optimal position to provide cervical or lumbar support. The desired arch, and in particular a parabolic arch, may be achieved by giving the tongue varying flexibility in the longitudinal or sagittal direction, for example, by varying the thickness or applying ribs.

The retaining mechanism may be in the form of a ratchet mechanism with a pawl engaging a toothed rack so as to offer defined gradations of curvature. Alternatively, a screw mechanism may be employed.

According to another aspect of the invention there is provided a cervical support with adjustable curvature, comprising a hard shell having a convex curvature in a longitudinal direction generally conforming to the curvature of a cervical spine, said hard shell forming a cradle in the lateral direction for accommodating a human head; a stiff resilient conformal tongue on an inner surface of said hard shell having one end hinged to the hard shell and an opposite end free to move in a longitudinal guide permitting forcible deformation of the tongue to create an arch with varying degrees of curvature; a retaining mechanism for locking said opposite end so as to retain said tongue in a deformed arch configuration with a selected degree of curvature; and a cushioning material located in said cradle for comfortably supporting the human head.

According to yet another aspect of the invention there is provided a lumbar support with adjustable curvature, comprising a hard shell having a convex curvature in a longitudinal direction generally conforming to the curvature of a lumbar spine, said hard shell conforming to the shape of a human lower back; a stiff resilient conformal tongue on an inner surface of said hard shell having one end hinged to the hard shell and an opposite end free to move in a longitudinal guide permitting forcible deformation of the tongue to create an arch with varying degrees of curvature; and a retaining mechanism for locking said opposite end so as to retain said tongue in a deformed arch configuration with a selected degree of curvature.

### BRIEF DESCRIPTION OF THE DRAWINGS

This invention will now be described in more detail, by way of example only, with reference to the accompanying drawings, in which:—

FIGS. 1a and 1b are schematic sections of a human body showing cervical and lumbar supports in different configurations;

FIGS. 2a and 2b are isometric view of the cervical support shown in FIGS. 1a and 1b;

FIGS. 3a and 3b are isometric longitudinal sectional views of the cervical support;

FIGS. 4a and 4b are side sectional views of the cervical support;

FIGS. 5a to 5c are side sectional views of the tongue;

FIGS. 6a to 6c show a front view of a cervical support with upper and lower cushions;

FIGS. 7a to 7d are side views of the cervical support;

FIG. 8a shows a rear view of the cervical support;

FIG. 8b shows a detail of FIG. 8; and

FIGS. 9a and 9b are isometric longitudinal sectional views of another embodiment of the cervical support.

### DETAILED DESCRIPTION OF PREFERRED EMBODIMENTS

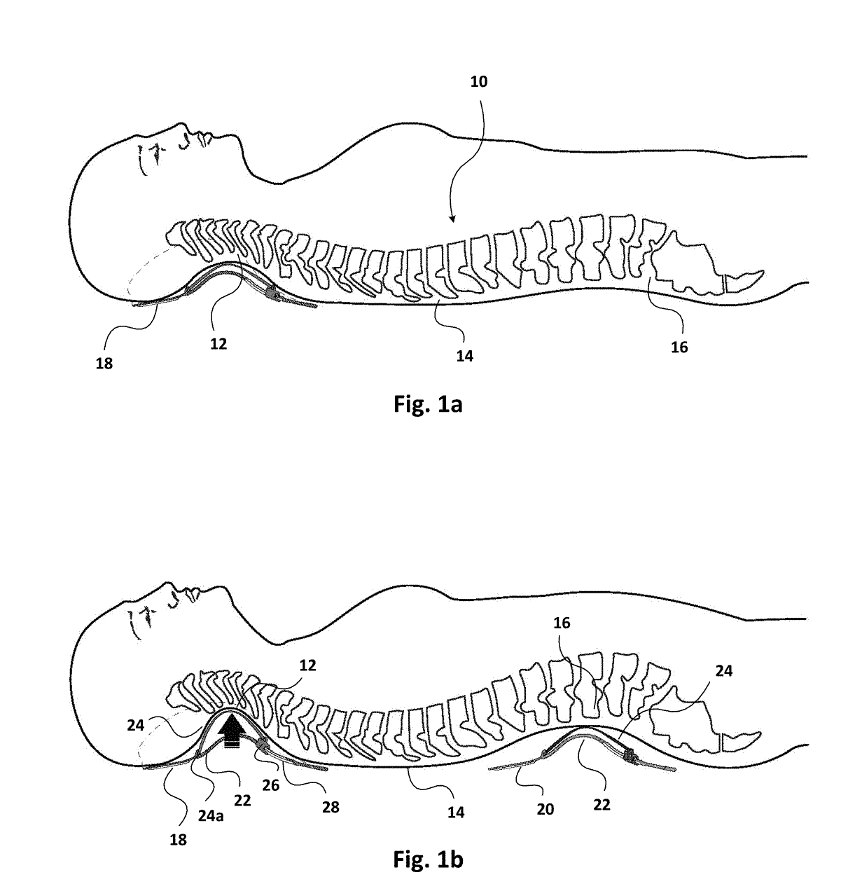

FIGS. 1a and 1b show the natural curvature of a human spine 10 in the unstressed state. The concave cervical spine 12 merges into the convex thoracic spine 14, which in turn merges into the lumbar spine 16. Loss of normal Lordosis in the cervical or lumbar spine, for example caused by prolonged poor posture, can lead to vertebral or disc damage, and chronic pain. Treatment involves correction of the deficient curvature by the use of an appropriately shaped support.

In FIGS. 1a and 1b, in accordance with one embodiment of the invention a spinal support with adjustable curvature, or cervical support, 18 is shown supporting the cervical spine 12. FIG. 1b also shows a spinal support with adjustable curvature, or lumbar support, 20 in accordance with an embodiment of the invention for supporting the lumbar spine 16.

The cervical support 18 comprises a hard or semi-rigid shell 22 having a convex curvature in the longitudinal or sagittal direction that generally, but not necessarily precisely, conforms to the concave curvature of the cervical spine 12. The hard shell 22 should have sufficient thickness to and rigidity to support the cervical spine or lumbar spine without significant deflection. In one non-limiting example, the hard shell may be made of polypropylene 5 mm thick.

A stiff, pliable, resilient deformable conformal tongue 24 lies on an inner surface of the hard shell 22. The tongue 24 can made of the same material as the hard shell 22. The tongue 24 should have sufficient stiffness to support the weight of a human head when shaped into an arch while affixed at the ends to the hard shell 22, but sufficiently pliable to allow the tongue 24 to be pushed into the arch shape by exerting manual pressure to one end.

The conformal tongue 24 is hinged at its upper end 24a to the hard shell 22 and guided at its lower end so that it may be forcibly be deformed into a parabolic shape by pushing toward the upper end 24a while being guided in a suitable guide. A ratchet mechanism locks the tongue 24 in position when the desired degree of curvature has been reached in one of a plurality of predetermined positions. Of course, it will be appreciated that in an alternative embodiment the upper end could be free and the lower end hinged to the hard shell 22, or both ends could be free while constrained to move in the longitudinal direction, in which case the tongue 24 would be pushed in from both ends.

The lumbar support 20 works in a similar manner to the cervical support. The curvature of the hard shell may be less to accommodate the shallower curvature of the lumbar spine 16 compared to the cervical spine 12. Also, as will be described, the cervical support 18 has more of a cradle shape in the transverse direction to accommodate the head, whereas the lumbar support 20 may be flatter so as to lie comfortably under the back of the patient.

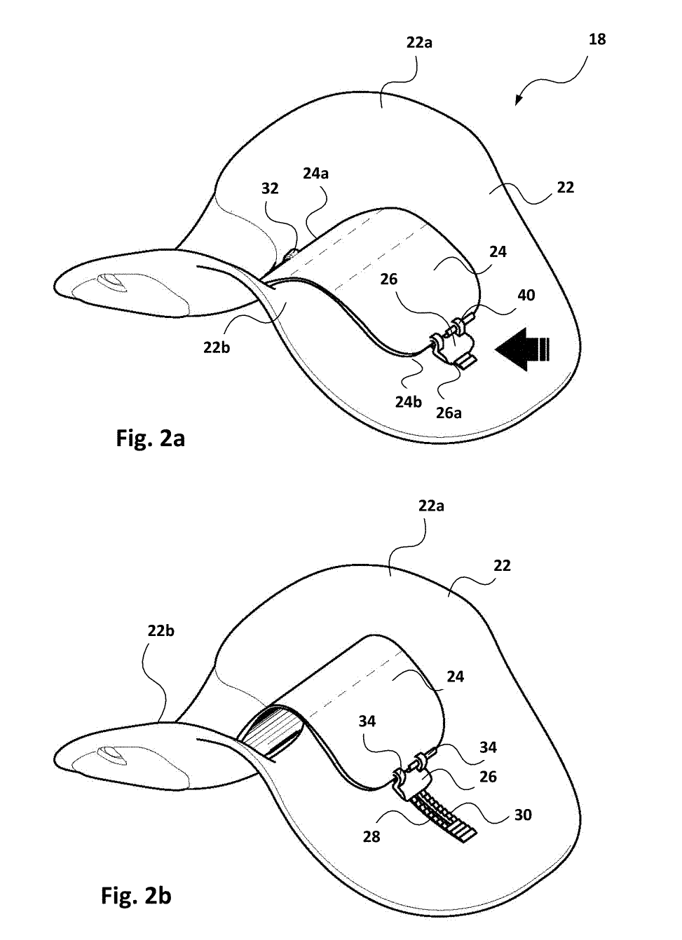

As shown in FIG. 2a the tongue 24, which is hinged at its upper end 24a to the hard shell 22, in its natural or unstressed state conforms generally to the shape of the hard shell 22 in the longitudinal or sagittal direction. In the transverse direction the hard shell has generally upwardly curved wing portions 22a 22b so that the hard shell forms a cradle for accommodating the head of a patient.

The lower end 24b of the tongue 24 is attached to a lug 26 retained in a longitudinal guide slot 28 formed in the hard shell 22. The lug 26 has a lower sharp edge forming a pawl 26a engaging the teeth of a toothed rack 30 bordering the guide slot 28 to form a ratchet mechanism.

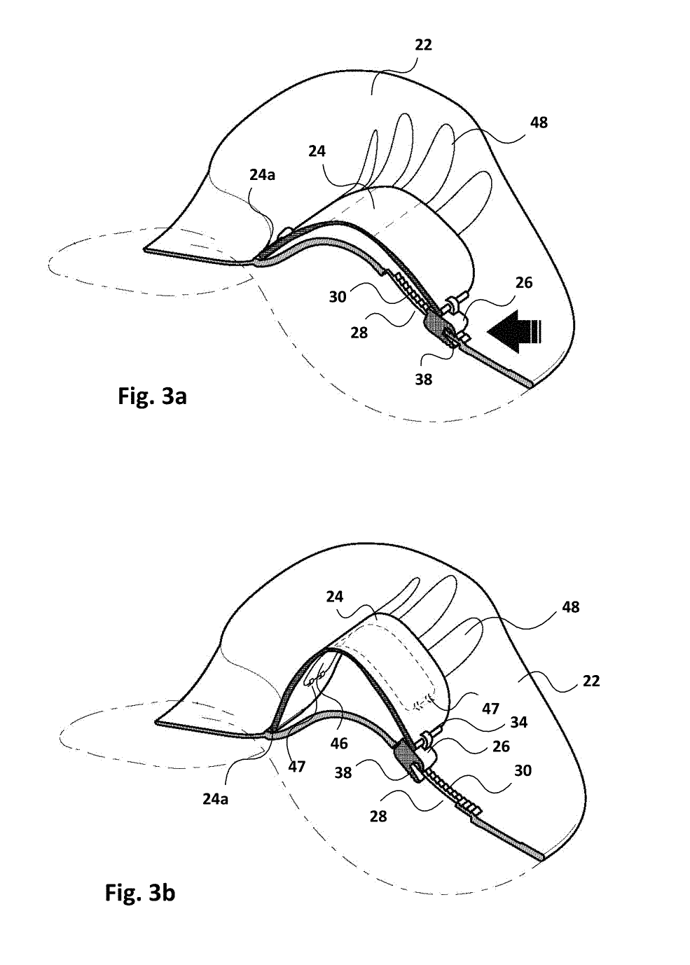

The application of a manual force on the lower edge 24b of the tongue 24, as shown in FIGS. 2a, 3a results in deformation of the tongue 24 into a parabolic shape as shown in FIGS. 2b,3b. The ratchet mechanism, formed by the pawl 26a engaging the teeth of the toothed rack 30, retains the tongue 24 in the desired position with the selected curvature. The various gradations of curvature of the tongue 24 after a manual force has been applied are determined by the locations of the teeth of the toothed rack 30.

As shown in FIGS. 3a and 2b, the lug 26 extends through the guide slot 28 so that it is retained in the guide slot 28, thereby ensuring the upward deflection of the tongue 24 as the lower end 24b is manually pushed upward toward the end 24a.

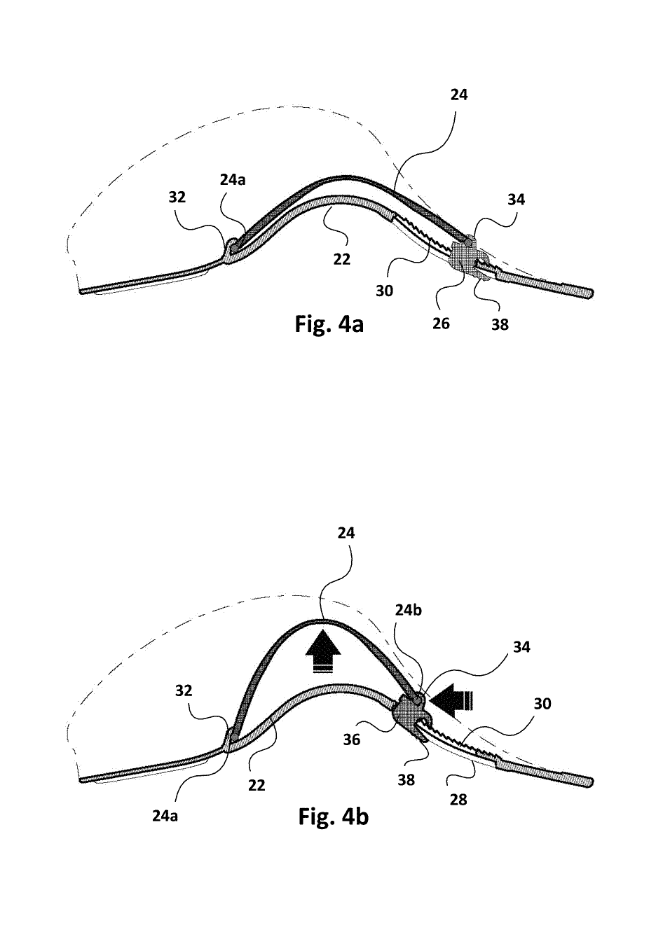

As shown in FIGS. 4a and 4b, the upper end 24a of the tongue 24 is clipped into a pair of laterally spaced brackets formed out of the hard shell 22, of which one 32, can be seen in FIGS. 2a, 4a, and 4b. The brackets 32 can be formed out of the hard shell 22 during injection molding. The upper end 24a of the tongue 24 may be made slightly bulbous to clip into the brackets 32 so as to form a hinge-like arrangement.

As shown in FIGS. 2a, 2b, 4a, 4b, the lug 26 also comprises a pair of spaced integrally formed brackets 34 into which the bulbous lower end 24b of the tongue 24 is clipped in a similar manner to the upper end 24a to form a hinge-like arrangement with the lug 26. The lug 26 has a central web 36 extending through the guide slot 28 merging into a tab 38 on the underside, which ensures that the lug 26 is constrained to move longitudinally within the guide slot 28.

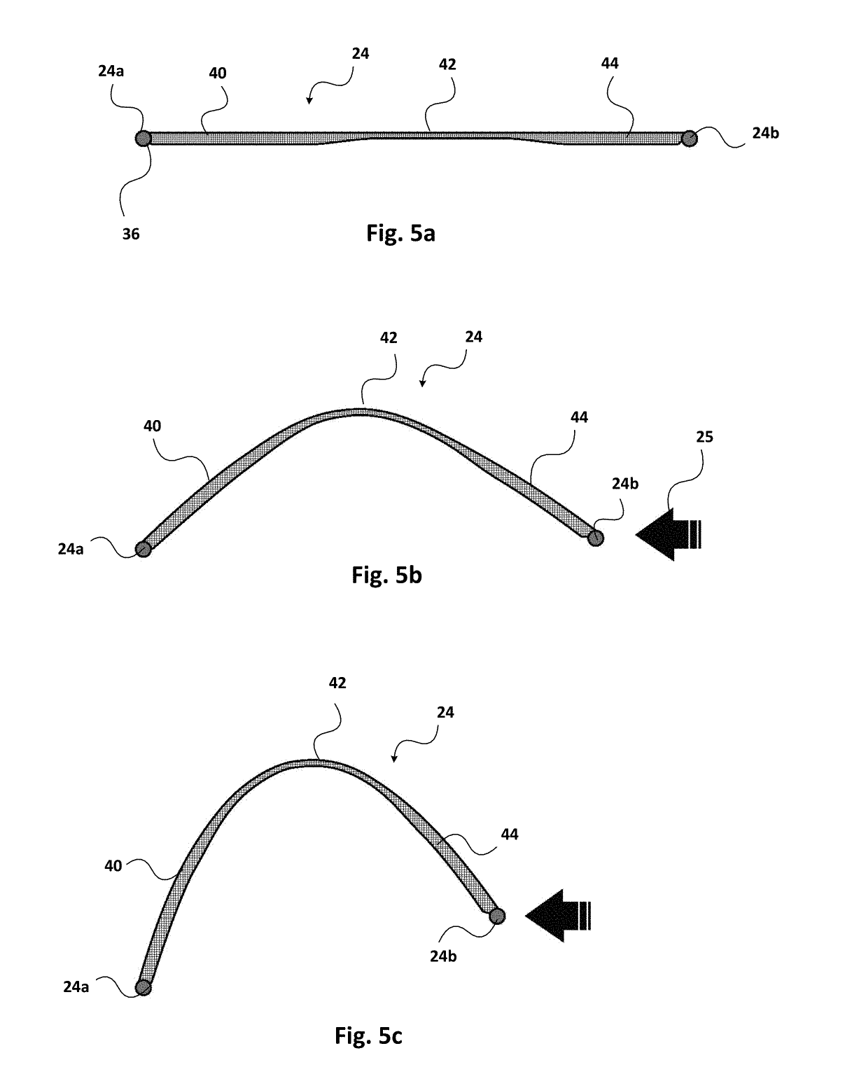

As shown in FIGS. 5a to 5c, the tongue 24 does not have a uniform thickness in the longitudinal direction, but has thicker outer regions 40, 44, and a thinner inner region 42 in order to promote deformation of the tongue into the desired parabolic or other specified shape when the tongue 24 is deformed to the application of a deformation force in the lateral direction shown by arrow 25. Typically the outer thicker regions 40, 44 are about twice as thick as the central thinner region 42. In one non-limiting example, wherein the tongue is made of polypropylene, the thicker regions are 0.120″ (3 mm) and the thinner central region is 0.060″ (1.5 mm). The profile of the tongue and the material used can be altered to suit. For example, in the case of a lumbar support the material would likely be thicker. As noted preferably the arch is a parabolic arch configured such that the vertex of the parabola is located at the optimal location for supporting the cervical spine 12.

The resilient tongue 24 should preferably be made of a pliable resilient material with shape memory, such as polypropylene plastic or acetal plastic. Preferably, the entire support should be made of thermoplastics, such as polypropylene plastic or acetal plastic, so that the support can be formed by injection molding. Alternatively, it could be fabricated from a resilient plastic such as such as polypropylene or acetal, composite plastic, or wood.

In one embodiment a steel leaf spring 46 (shown in FIG. 3b) may be fitted on the underside of the tongue 24 to help the tongue 24 spring back into position when the pawl 26a is released from the toothed rack 30 by pressure on the tab 38 on the underside of the lug 26. The leaf spring 46 may be held in place by brackets 47 molded into the hard shell 22 in a similar manner to which the ends 24a, 24b of the tongue 24 are held in place on the hard shell 22. The leaf spring 46 could alternatively be replaced by a coil spring.

Each side of the tongue 24 may be extended by outwardly projecting fingers 48 conforming to the saddle shape of the hard shell 22.

Of course, it will be appreciated that it would also be possible to deflect both ends of the tongue 24.

Rather than varying the thickness of the tongue to create the desired curvature, another solution would be to add ribs of varying thickness running perpendicular to the curve.

It will also be appreciated that the lug 26 that forms the pawl 26a could be molded integrally with the tongue 24.

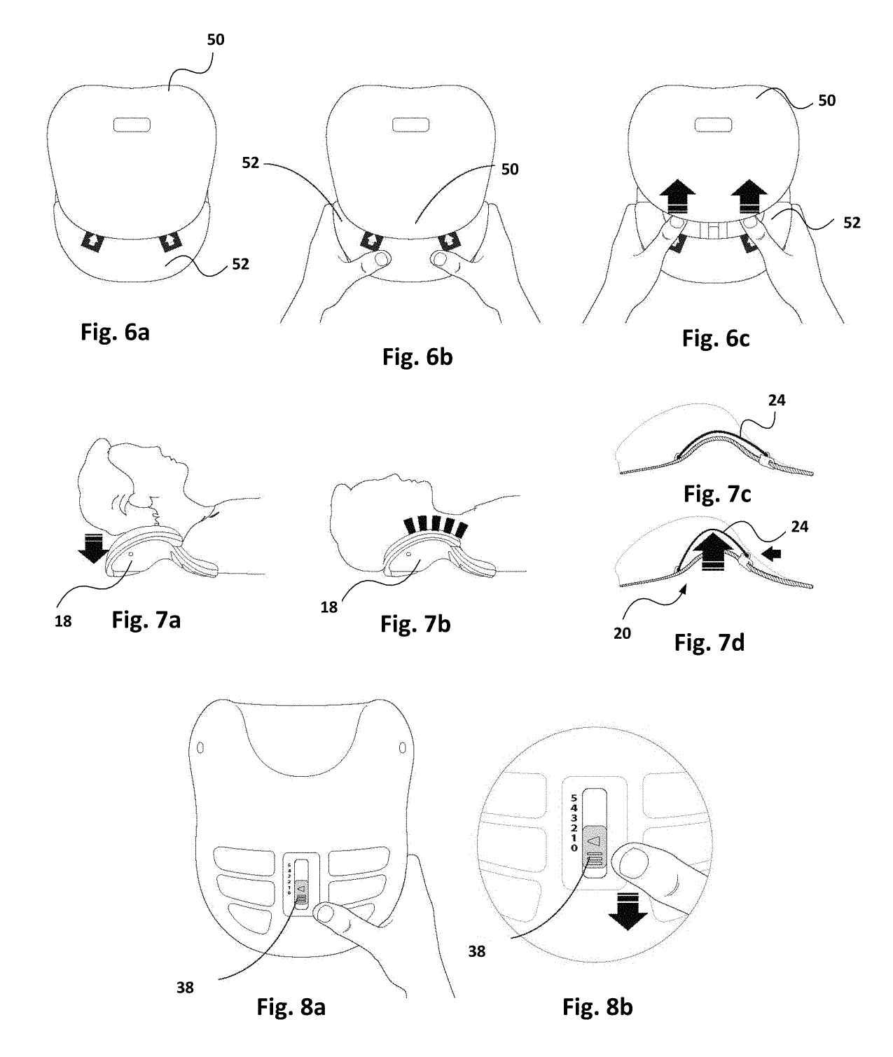

The cervical support shown in FIGS. 6a to c includes removable upper head cushion 50 and a removable lower collar cushion 52 attached to the hard shell 22 by means of loop and hook fasteners, such as Velcro™. The cushion is preferably made of a breathable mesh to stay cool and fresh.

FIGS. 6a, 7b and 7c show the relaxed position with minimal arch. When it is desired to increase the curvature and arch height thumb pressure is applied to the lower end of the tongue 24 as shown in FIG. 6b. This causes the lug 26 to ratchet up the rack and retain the tongue 24 in the deformed position as shown in FIG. 7d, thereby increasing the depth of the arch and creating a pressure point on the cervical spine where desired. The tab 38 on the rear side of the hard shell 22 has an arrow which points to index marks on hard shell 22 showing the degree of deformation of the tongue 24.

When it is desired to return the cervical support to the relaxed position thumb pressure is applied to the tab 38, thereby releasing the lug 26 from the teeth of the rack 30 and allowing the resilient tongue 24 to return to the relaxed position owing to its natural memory and assisted if desired by the optional leaf spring 46.

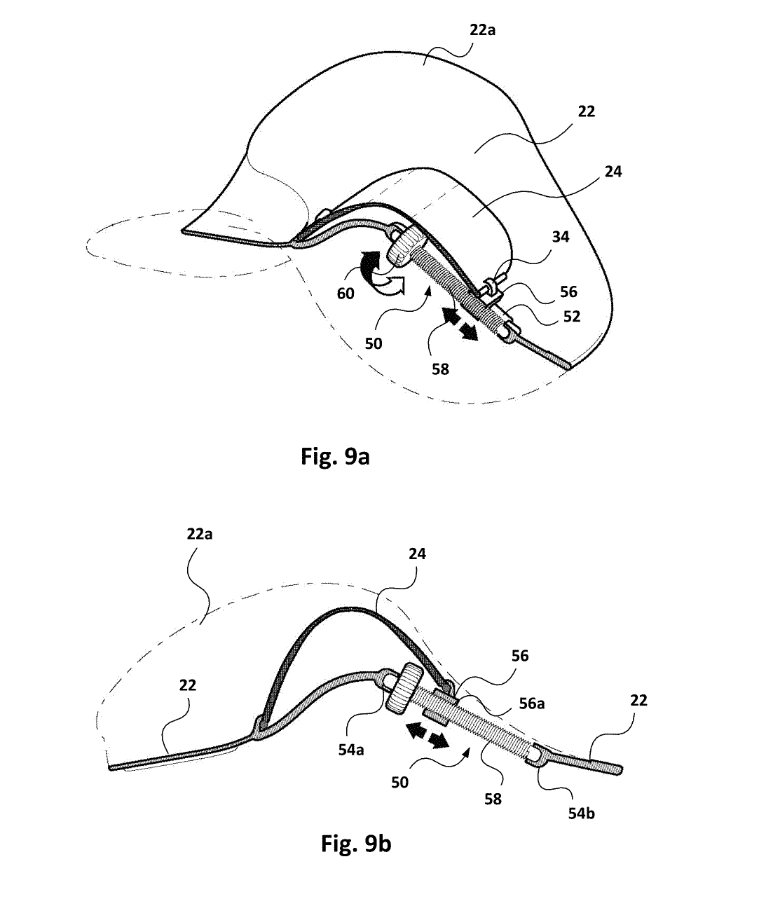

Other mechanisms can be employed to retain the lower end of the tongue in the desired position. For example, in an alternative embodiment shown in FIGS. 9a and 9b the ratchet mechanism is replaced by a screw mechanism 50 retained in a longitudinal slot 52 formed in the hard shell 22 between molded end cups 54a, 54b so as to permit rotation thereof.

The lug 56 has a threaded bore 56a riding on a threaded rod 58. A knurled knob 60 is affixed to the end of the threaded rod 58. Turning the knob 60 results in the lug 56 riding up and down the threaded rod 58, thereby changing the curvature of the tongue 24.

The screw mechanism provides a mechanical advantage, which may be easier for certain users to operate, especially if a stiffer tongue 24 is employed.

1. A spinal support with adjustable curvature, comprising:
a hard shell having a convex curvature in a longitudinal direction generally conforming to a concavely curved region of a human spine;
a stiff resilient conformal tongue on an inner surface of said hard shell having one end hinged to the hard shell and an opposite end free to move in a longitudinal guide permitting forcible deformation of the tongue to create an arch with varying degrees of curvature; and
a retaining mechanism for locking said opposite end so as to retain said tongue in a deformed arch configuration with a selected degree of curvature.

2. The spinal support of claim 1, wherein said tongue has varying degrees of flexibility in the longitudinal direction.

3. The spinal support of claim 2, wherein said tongue has a varying thickness in the longitudinal direction.

4. The spinal support of claim 3, wherein said tongue is configured so that in the deformed configuration said arch is a parabolic arch.

5. The spinal support of claim 2, further comprising a spring attached to a rear side of said tongue to assist in restoring said tongue to its relaxed condition upon release of said retaining mechanism.

6. The spinal support of claim 5, wherein said spring is a leaf spring.

7. The spinal support of claim 1, wherein said retaining mechanism comprises a ratchet mechanism between said hard shell and said tongue, said ratchet mechanism permitting the tongue to be retained with different selected degrees of curvature.

8. The spinal support of claim 8, wherein said ratchet mechanism comprises a lug attached to said tongue and constrained to move in a guide slot in the hard shell, said lug having a pawl engaging a toothed rack.

9. The spinal support of claim 8, wherein said lug includes a tab on a rear side of said hard shell for releasing said pawl from said toothed rack to restore said tongue to the relaxed position.

10. The spinal support of claim 1, wherein said retaining mechanism comprises a screw mechanism between said hard shell and said tongue, said screw mechanism permitting the tongue to be retained with different degrees of curvature.

11. The spinal support of claim 10, wherein said screw mechanism comprises a lug attached to said tongue riding on a threaded rod rotatably mounted in a slot formed in the hard shell.

12. The spinal support of claim 1, which is a cervical support, and wherein said hard shell forms a head-receiving cradle, with a cushioning material located in said head-receiving cradle.

13. The spinal support of claim 10, wherein said tongue has lateral fingers conforming extending into wing portions of said cradle underlying said cushioning material.

14. The spinal support of claim 1, which is a lumbar support and said hard shell is shaped to conform to a lower back region of a human.

15. The spinal support of claim 1, wherein said hard shell and said tongue are injection molded from polypropylene.

16. A cervical support with adjustable curvature, comprising:
a hard shell having a convex curvature in a longitudinal direction generally conforming to the curvature of a cervical spine, said hard shell forming a cradle in the lateral direction for accommodating a human head;
a stiff resilient conformal tongue on an inner surface of said hard shell having one end hinged to the hard shell and an opposite end free to move in a longitudinal guide permitting forcible deformation of the tongue to create an arch with varying degrees of curvature;
a retaining mechanism for locking said opposite end so as to retain said tongue in a deformed arch configuration with a selected degree of curvature; and
a cushioning material located in said cradle for comfortably supporting the human head.

17. The spinal support of claim 16, wherein said tongue is configured so that in the deformed configuration said arch is a parabolic arch.

18. The spinal support of claim 16, wherein said retaining mechanism is selected from the group consisting of: a ratchet mechanism and a screw mechanism, for locking said tongue in a selected arch position.

19. A lumbar support with adjustable curvature, comprising:
a hard shell having a convex curvature in a longitudinal direction generally conforming to the curvature of a lumbar spine, said hard shell conforming to the shape of a human lower back;
a stiff resilient conformal tongue on an inner surface of said hard shell having one end hinged to the hard shell and an opposite end free to move in a longitudinal guide permitting forcible deformation of the tongue to create an arch with varying degrees of curvature; and
a retaining mechanism for locking said opposite end so as to retain said tongue in a deformed arch configuration with a selected degree of curvature.

20. The spinal support of claim 19, wherein said retaining mechanism is selected from the group consisting of: a ratchet mechanism or a screw mechanism for locking said tongue in a selected arch position.
